(19) United States
(12) Patent Application Publication
(10) Pub. No.: US 2020/0107741 A1
(43) Pub. Date: Apr. 9, 2020

(54) ELECTRODES AND ELECTRODE UNITS FOR GATHERING ELECTROENCEPHALOGRAPHIC DATA

(71) Applicant: The Nielsen Company (US), LLC, New York, NY (US)

(72) Inventors: Kashyap Vinodchandra Fruitwala, Berlin (DE); Mehran Tirooni, Berlin (DE); Mohammad Moradi, Berlin (DE); Batia Bertho, Berlin (DE); Carlos Schreib, Berlin (DE)

(21) Appl. No.: 16/154,980

(22) Filed: Oct. 9, 2018

(51) Int. Cl. A61B 5/0478; A61B 5/00

(52) U.S. Cl. CPC A61B 5/0478; A61B 5/6803

(57) ABSTRACT

Electrodes and electrode units for gathering electroencephalographic data are described herein. An example electrode unit includes a housing and an electrode coupled to and extending from the housing. The electrode includes a first shell, a second shell retractable relative to the first shell, a third shell retractable relative to the second shell, and an electrode pin retractable relative to the third shell. The first shell, the second shell, the third shell, and the electrode pin are configured in a telescoping arrangement and moveable between an expanded state and a compressed state. The electrode includes a spring to bias the second shell outward from the first shell, the third shell outward from the second shell, and the electrode pin outward from the third shell to contact skin of a subject.

### FIELD OF THE DISCLOSURE

This disclosure relates generally to neurological and physiological monitoring and, more particularly, to electrodes and electrode units for gathering electroencephalographic data.

### BACKGROUND

Electroencephalography (EEG) involves measuring and recording electrical activity corresponding to neural processes in the brain. EEG data is typically measured using a plurality of electrodes placed on the scalp of a subject to measure voltage fluctuations resulting from this electrical activity within the neurons of the brain.

### BRIEF DESCRIPTION OF THE DRAWINGS

FIG. 1 is a side view of an example headset with which example electrodes and/or example electrode units disclosed herein may be used.

FIG. 2 illustrates a section of an example band of the example headset of FIG. 1.

FIG. 3 is a side view of an example electrode unit having an example electrode that may be used with the example headset of FIG. 1.

FIG. 4 is a cross-sectional view of the example electrode unit of FIG. 3 taken along line A-A of FIG. 3.

FIG. 5 is a cross-sectional view of the example electrode of the example electrode unit of FIGS. 3 and 4 in a compressed or retracted state.

FIG. 6 is an exploded perspective view of the example electrode unit of FIGS. 3 and 4.

FIG. 7 is a cross-sectional view of an example of the electrode of FIGS. 4-7 having additional springs.

FIG. 8 is a cross-sectional view of an example of the electrode of FIGS. 4-7 having another alternative spring configuration.

FIG. 9 is a side view of an example electrode unit having an example electrode that may be used with the example headset of FIG. 1.

FIG. 10 is a cross-sectional view of the example electrode unit of FIG. 9 taken along line B-B of FIG. 9.

FIG. 11 is a cross-sectional view of the example electrode of the example electrode unit of FIGS. 9 and 10 in a compressed or retracted state.

FIG. 12 is an exploded side view of the example electrode unit of FIG. 9.

FIG. 13 is an exploded perspective view of the example electrode unit of FIG. 9.

FIG. 14 is a side view of an example electrode tip having a flat shape that may be implemented in connection with any of the example electrodes disclosed herein and/or any other electrode.

FIG. 15 is a side view of an example electrode tip having a hump shape that may be implemented in connection with any of the example electrodes disclosed herein and/or any other electrode.

FIG. 16 is a side view of an example electrode tip having a hollow shape that may be implemented in connection with any of the example electrodes disclosed herein and/or any other electrode.

The figures are not to scale. Instead, the thickness of the layers or regions may be reduced or enlarged in the drawings. In general, the same reference numbers will be used throughout the drawing(s) and accompanying written description to refer to the same or like parts.

### DETAILED DESCRIPTION

Disclosed herein are example telescoping electrodes for obtaining EEG signals from a head of a subject. An example telescoping electrode disclosed herein includes an electrode pin and a body having one or more portions (e.g., shells). In some examples, the electrode includes a first shell, a second shell retractable relative to the first shell, a third shell retractable relative to the second shell, and an electrode pin retractable relative to the third shell. The electrode pin and the shells configured or arranged in a telescoping arrangement. The telescoping arrangement enables the electrode to compress or expand between an expanded state (having a longer length) and a compressed state (having a shorter length). The example electrode may include a spring that biases the electrode pin outward and, thus, biases the electrode to the expanded state.

The example telescoping electrode may be used with a headset worn on a head of the subject. In particular, the electrode can be coupled to a band or other structure of a headset in a position where the electrode extends downward from the headset toward the scalp of the subject. In the extended state, the telescoping electrode is relatively long, which enables the electrode to easily penetrate longer hair, thicker hair, and/or curly hair including afro-texture hair. For example, when a headset is disposed on a head of a subject with longer, thicker, and/or curly hair, the headset is disposed relatively far from the scalp due to the subject's hair. Using a longer telescoping electrode enables the electrode tip to protrude through the hair and contact the scalp of the subject for obtaining EEG signals. As such, the example telescoping electrode can obtain better quality EEG signals than shorter electrodes that do not extend through the hair. Further, the example telescoping electrode may be used with subjects having short hair or no hair. In particular, as the headset is moved closer to the subject's head, the telescoping electrode compresses or shortens. Thus, the example telescoping electrode and be used with different types of hair. Further, this compression avoids poking the scalp while ensuring good contact with the scalp. As such, example telescoping electrodes disclosed herein obtain better quality signals while increasing comfort for a variety of types, textures, and/or lengths of hair.

Example telescoping electrodes disclosed herein may be part of an electrode unit including a housing. In such an example, the electrode is coupled to and extends outward from the housing. The electrode is compressible or retractable into the housing. The housing may be constructed of plastic, for example, to insulate the electrode from interference and other signals in the atmosphere. In other examples, other insulating materials or combination of materials may be used. In some examples, the electrode unit includes a connector (e.g., a magnetic ring or a metal ring) to magnetically couple the electrode unit to a headset. In other examples, the electrode unit can be coupled with a mechanical fastener to the headset such as, for example, a threaded connection or a friction fit.

Another example electrode unit disclosed herein includes a housing and an electrode that is coupled to and moveable into a cavity of the housing. In some examples, a spring (e.g., a conceal compression or extension spring) is disposed within the cavity and biases the electrode outward from the housing. In some examples, the electrode unit includes a connector to magnetically couple the electrode unit to a headset.

Also disclosed herein are example electrode tip shapes for obtaining better quality signals while increasing comfort. An example electrode tip having a hump shape is disclosed herein. In this design, a hump extends from an end surface of the electrode tip. The hump has a smaller cross-section than the rest of the electrode. The hump is capable of penetrating through (i.e., compressing into) the first layer of skin, which enables the electrode to obtain better quality signals (because the electrode is closer to the source of the EEG signals and makes greater contact with the tissue). However, the larger diameter portion of the electrode prevents the hump from extending too far into the skin. Thus, the example hump shape enables lower impedance without compromising comfort. Another example electrode tip shape is a hollow shape. In this design, a recess extends into a center of an end surface of the electrode tip. As a result, a ring-shaped end surface is formed. This ring shaped end surface has a flat section that covers a larger area with less surface contact. This type of design is more comfortable for persons with short hair or no hair.

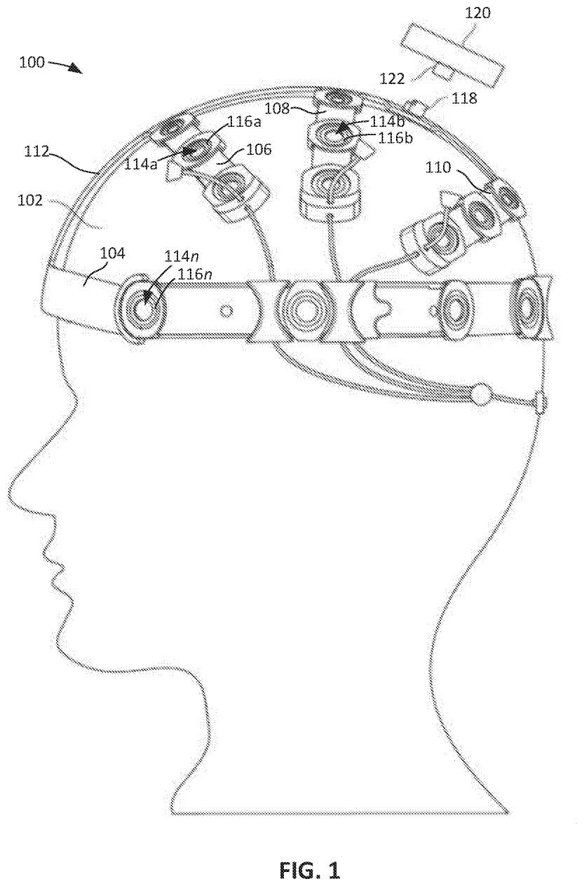

Turning now to the examples illustrated in the figures, FIG. 1 shows an example headset 100 with which example electrodes and/or example electrode units disclosed herein may be implemented. The example headset 100 may be worn on a head 102 of a subject (e.g., a person). One or more electrodes and/or electrode units may be coupled to the headset 100 and used to gather EEG signals from the head 102 of the subject. As used herein, a subject may be any person, user, viewer, participant and/or panelist. A panelist may be, for example, a user registered on a panel maintained by a ratings entity (e.g., an audience measurement company) that owns and/or operates a ratings entity subsystem. Traditionally, audience measurement entities (also referred to herein as “ratings entities”) determine demographic reach for advertising and media programming based on registered panel members. That is, an audience measurement entity enrolls people that consent to being monitored into a panel. During enrollment, the audience measurement entity receives demographic information from the enrolling people so that subsequent correlations may be made between advertisement/media exposure to those panelists and different demographic markets. People become panelists via, for example, a user interface presented on the media device (e.g., via a website). People become panelists in additional or alternative manners such as, for example, via a telephone interview, by completing an online survey, etc. Additionally or alternatively, people may be contacted and/or enlisted using any desired methodology (e.g., random selection, statistical selection, phone solicitations, Internet advertisements, surveys, advertisements in shopping malls, product packaging, etc.).

In another example, the subject may be a patient. For example, the headset 100 may be used by a patient for monitoring (e.g., in-home or at a medical facility), treatment and/or diagnosis of medical conditions, to detect life-threatening situations, to ascertain patient compliance with a prescribed medical regime and/or other suitable applications. The headset 100 is usable for many type(s) of patients with many type(s) of conditions including cardiac arrhythmia, epileptic seizures, stroke, small vessel disease, dementia, memory loss, Alzheimer's, glucose monitoring, blood pressure, hypertonia, cognitive decline, depression and/or other conditions. Other physiological conditions, psychiatric conditions, disease progression, disease intervention effectiveness and/or developmental disorders are also monitorable such as, for example, bipolar disorder, schizophrenia, attention deficit hyperactivity disorder (ADHD) and/or autism. In still other examples, the headset 100 may be used for other purposes, such as controlling aspects of a game or other entertainment, providing data as part of a fitness regime, controlling remote devices, and/or multiple other uses.

The headset 100 may include one or more bands to which one or more electrodes and/or electrode units can be coupled. In this example, the headset 100 includes a head band 104 that fits around the head 102 of the subject. The example headset 100 further includes a first band 106, a second band 108, and a third band 110 that are positioned to extend over the head 102 of subject from the left side to the right side of the head 102. In the illustrated example, the first, second, and third bands 106, 108, 110 are coupled to the head band 104 by a midline band 112. The midline band 112 extends from the head band 104 and is positioned to extend over the head 102 of the subject from the front to the rear of the head 102, or from the back to the front of the head 102 (e.g., along the midline), depending on the orientation the headset 100 is worn. In other examples, the headset 100 may include more or fewer bands. The number of bands, lengths of bands, shapes of bands, orientation of bands, etc. may be based on the desired number of channels from which EEG signals are to be gathered and/or the desired locations of measurement. Also, in some examples, headsets with a netting or a cap or other headset configurations without bands may also be used. Further still, in some examples, an electrode may be used without a headset and may be incorporated into another wearable device (e.g., a watch, a chest band, etc.) and/or otherwise used to gather signals from a subject.

Electrodes may be coupled to the headset 100 and used to obtain EEG signals from the head 102 of the subject. Example electrodes and electrode units having electrodes are disclosed in further detail herein. In some examples, electrodes may be coupled to each of the head band 104, the first band 106, the second band 108, the third band 110, and the midline band 112. In other examples, only certain ones of the bands 104-112 may carry electrodes. The electrodes may be coupled to the headset 100 at apertures formed in one or more of the bands 104-112. In particular, the headset 100 includes a plurality of apertures 114a-114n (e.g., an opening, a hole, etc.) to receive electrodes. Any number (n) of apertures may be employed. The apertures 114a-114n may be formed in any of the head band 104, the first band 106, the second band 108, the third band 110 and/or the midline band 112, depending on the desired location of electrodes. When an electrode is coupled to the headset 100, the electrode extends through a corresponding aperture to engage the head 102 of the subject.

To connect electrodes to the bands 104-112, the example headset 100 includes a plurality of connectors 116a-116n disposed adjacent to (e.g., around) corresponding ones of the apertures 114a-114n. In some examples, one or more of the connectors 116a-116n are implemented as metal rings, and the electrodes (or electrode units) may include a magnetic ring to couple the respective electrode (or electrode unit) to respective ones of the connectors 116-116n. In other examples, one or more of the connectors 116a-116n may be implemented as magnetic rings, and the electrodes (or electrode units) may include a metallic structure (e.g., a metal ring) to couple the respective electrode (or electrode unit) to respective ones of the connectors 116-116n. The magnetic attraction between an the electrode (or an electrode unit) and a corresponding one of the connectors 116a-116n holds the electrode to the headset 100. In other examples, other types of connectors (e.g., a hook and loop fastener) may be implemented.

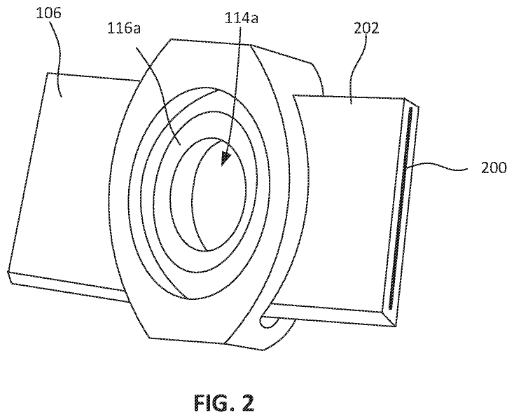

In some examples, to communicate the signal(s) gathered by the electrodes on the headset 100 to a computing device for analysis, the headset 100 includes one or more printed circuit boards (PCBs) (e.g., a substrate on which circuitry may be mounted and/or printed) disposed within one or more bands 104-112. For example, silicone or another material may be molded around a PCB to form one or more of the bands 104-112 of the headset 100. The PCB may be flexible and may include traces or wires to form circuitry. For example, referring briefly to FIG. 2, FIG. 2 shows a section of the first band 106 and with the first aperture 114a and the first connector 116a. A PCB 200 is molded within a body 202 of the first band 106. The first connector 116a is electrically coupled to the PCB 200. When an electrode or electrode unit is coupled to the first connector 116, signal(s) gathered by the electrode are transmitted through the first connector 116a to the PCB 200. Similarly, the other connectors 116b-116n may be in circuit with one or more PCBs disposed within the respective bands 104-112.

In the illustrated example of FIG. 1, the headset 100 includes an electrical connector 118 to which a processor 120 (e.g., a controller, a microprocessor, a central processing unit (CPU), an application-specific integrated circuit (ASIC) or the like) can be connected. The PCB(s) (e.g., the PCB 200) of the headset 100 may communicatively couple one or more electrode(s) positioned in respective ones of the apertures 114a-114n to the electrical connector 118. As such, signals gathered by the electrodes are transmitted to the processor 120 for collection and/or analysis. The example processor 120 includes an electrical connector 122 that can be plugged into the electrical connector 118. In some examples, the processor 120 conditions the signals, filters/attenuates noise, provides additional signal processing and/or signal analysis and/or outputs data to an external device. In some examples, the processor 120 includes a transmitter to transmit signals gathered by the electrodes and/or data based on such signals. In other examples, rather than transmitting the EEG signals through PCBs in the headset 100, one or more wires may be electrically coupled to the respective electrodes and/or the connector. The wire(s) can be connected to the processor 120 and/or a remote computing device (e.g., a desktop computer adjacent the user) for signal collection and/or analysis.

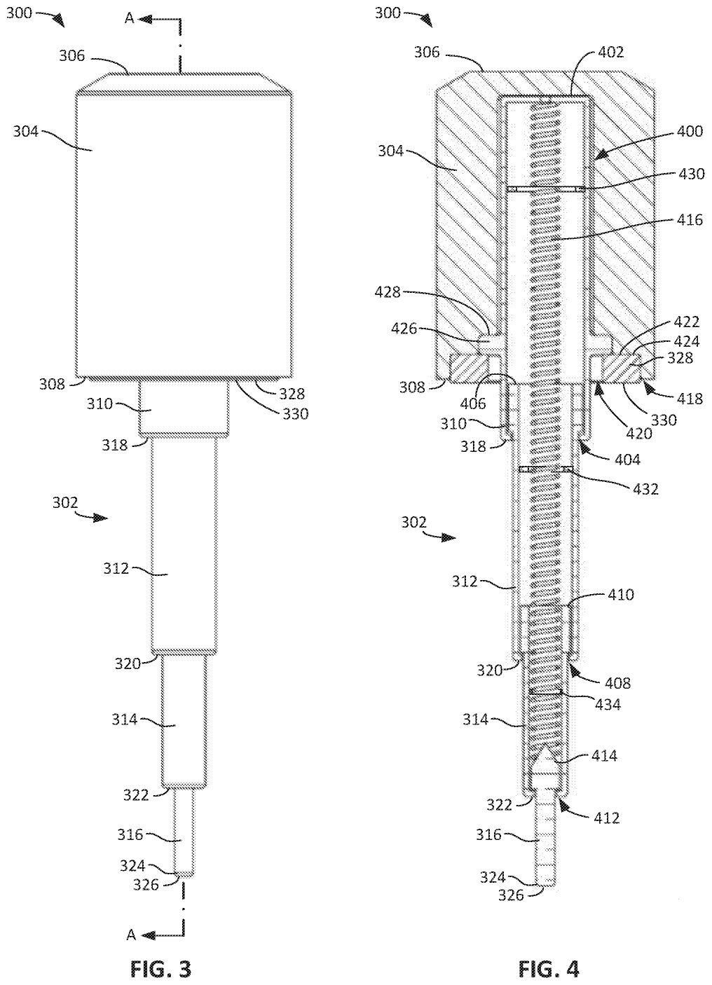

FIGS. 3 and 4 illustrate an example electrode unit 300 having an example electrode 302 that may be used with the example headset 100 of FIG. 1 and/or another headset or wearable device. FIG. 4 is a cross-sectional view of the example electrode unit 300 taken along line A-A in FIG. 3. The electrode unit 300 may be coupled to one of the bands 104-112 of the headset 100, for example. In some examples, multiple ones of the electrode unit 300 may be coupled to and used with the example headset 100.

In the illustrated example of FIGS. 3 and 4, the example electrode unit 300 includes a housing 304. The electrode 302 is coupled to and extends outward from the housing 304. The housing 304 has a first side 306 (e.g., a top side) and a second side 308 (e.g., a bottom side) opposite the first side 306. As shown in FIG. 4, the electrode 302 is at least partially disposed within a cavity 400 formed in the housing 304, and the electrode 302 extends outward from the second side 308 of the housing 304. The electrode unit 300 may be disposed on one of the bands 104-112 of the headset 100 at one of the apertures 114a-114n. For example, the electrode unit 300 may be coupled to the first band 106 at the first aperture 114a shown in FIG. 2. When the electrode unit 300 is disposed on the first band 106, for example, the second side 308 the electrode 302 is disposed the first band 106 and the electrode 302 extends through the first aperture 114a to engage the head 102 of the subject. The electrode unit 300 may be coupled to the first band 106 via the first connector 116a, as disclosed in further detail herein.

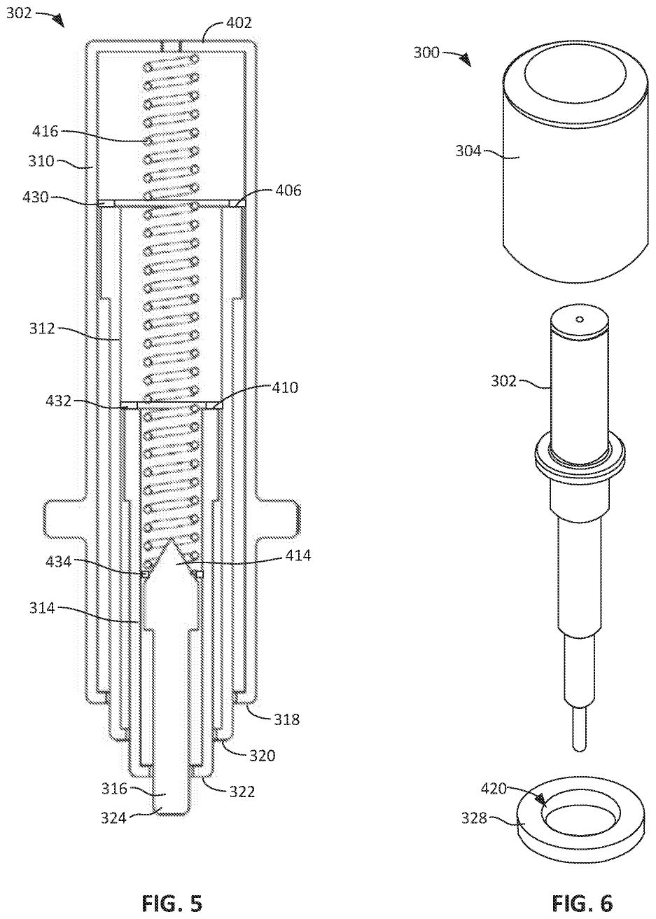

In this example, the electrode 302 is a telescoping electrode having two or more sections or portions that retract and/or expand to form a telescoping arrangement. For example, the electrode 302 of the illustrated example includes a first shell 310, a second shell 312 that is retractable into or extendible from the first shell 310, a third shell 314 that is retractable into or extendible from the second shell 312, and an electrode pin 316 (which may be referred to as a plunger) that is retractable into or extendible from the third shell 314. The first shell 310, the second shell 312, the third shell 314, and the electrode pin 316 are configured or arranged in telescoping arrangement and are moveable (e.g., compressible and expandable) between an expanded or extended state, as shown in FIGS. 3 and 4, and a compressed or retracted state, as shown in FIG. 5 (discussed in further detail herein).

In the illustrated example of FIGS. 3 and 4, the first shell 310 is disposed within the cavity 400 of the housing 304 and extends outward from the second side 308 of the housing 304. The first shell 310 has a first end 402 (FIG. 4) (e.g., a top end) and a second end 318 (e.g., a bottom or distal end) opposite the first end 402. The second end 318 of the first shell 310 is disposed outward from the housing 304. An opening 404 (FIG. 4) is formed in the second end 318 of the first shell 310. The second shell 312 is slidably disposed within the first shell 310. The second shell 312 is moveable into and outward from the second end 318 through the opening 404 (FIG. 4).

Similarly, the second shell 312 has a first end 406 (FIG. 4) (e.g., a top end) and a second end 320 (e.g., a bottom or distal end) opposite the first end 406. An opening 408 (FIG. 4) is formed in the second end 320 of the second shell 312. The third shell 314 is slidably disposed within the second shell 312 and moveable into and outward from the second end 320 through the opening 408 (FIG. 4).

The third shell 314 has a first end 410 (FIG. 4) (e.g., a top end) and a second end 322 (e.g., a bottom or distal end) opposite the first end 410. An opening 412 (FIG. 4) is formed in the second end 322 of the third shell 314. The electrode pin 316 is slidably disposed within the third shell 314 and moveable into and outward from the second end 322 through the opening 412. The electrode pin 316 has a first end 414 (FIG. 4) (e.g., a top end) and a second end 324 (which may be referred to as a bottom end or an electrode tip 324) opposite the first end 414. The tip 324 is to contact the skin of the subject.

As shown in FIG. 4, the first end 406 of the second shell 312 has a larger diameter than the opening 404 in the second end 318 of the first shell 310, which prevents the second shell 312 from being completely ejected or separated from the first shell 310. Similarly, the first end 410 of the third shell 314 has a larger diameter than the opening 408 in the second end 320 of the second shell 312, which prevents the third shell 314 from being completely ejected or separated from the second shell 312. Likewise, the first end 414 of the electrode pin 316 has a larger diameter than opening 412 in the second end 322 of the third shell 314, which prevents the electrode pin 316 from being ejected from the second end 322 of the third shell 314. In this example, the first, second, and third shells 310, 312, 314 are hollow or tubular while the electrode pin 316 is solid.

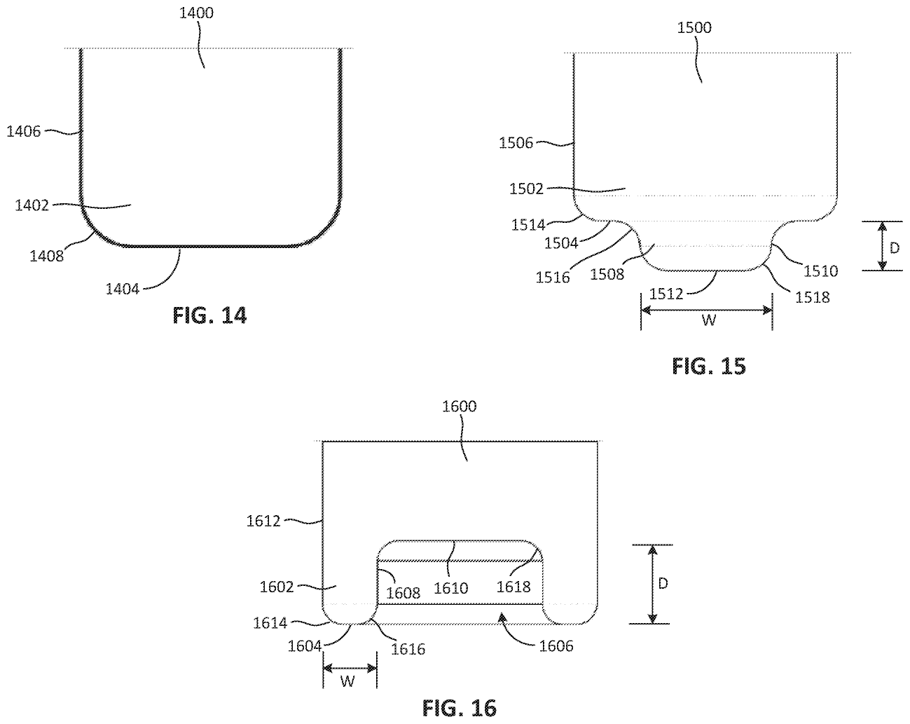

The tip 324 of the electrode pin 316 is to contact the scalp or be disposed near the scalp of the subject to obtain EEG signals from the head 102 of the subject. In the illustrated example, the tip 324 has an end surface 326 that is flat (e.g., perpendicular to a side surface of the electrode pin 316). However, in other examples, the tip 324 may be shaped differently. Other example electrode tip shapes are disclosed in further detail in connection with FIGS. 14-16.

The example electrode 302 is compressible or expandable between an expanded or extended state, as shown in FIGS. 3 and 4, and a compressed or retracted state, as shown in FIG. 5. As shown in FIG. 5, the electrode tip 316 has been moved upward into the third shell 314, the third shell 314 has been moved upward into the second shell 312, and the second shell 312 has been moved upward into the first shell 310. The term “upward” is used here relative to the image shown in FIGS. 3-5. In some examples, the electrode 302 may be positioned on a side of a head and the “upward” movement into the shells is actually a lateral movement or a combination of lateral and upward movement. In addition, in some examples, the electrode 302 may be position on a lower rear of the head of the subject (e.g., near or under the inion). In this example, the “upward” movement into the shell is a downward movement or a combination of a lateral and downward movement.

With the movements of the shells, the length of the electrode 302 can be decreased. In the example of FIG. 5, the electrode pin 316 does not extend fully into the third shell 314. Instead, the tip 324 remains outward from the second end 322 of the third shell 314. In some examples, this ensures the electrode pin 316 can make proper contact and the scalp. Also, in this example, in the compressed state of FIG. 5, the second end 322 of the third shell 314 remains outward from the second end 320 of the second shell 312, and the second end 320 of the second shell 312 remains outward from the second end 318 of the first shell 310. In some examples, to prevent the shells 310, 312, 314 and the electrode tip 316 from retracting fully into each other, one or more stops may be used. For example, as shown in FIGS. 4 and 5, a first stop 430 is disposed on (coupled to) an inner surface of the first shell 310. In this example, the first stop 430 is implemented as a ring. When the second shell 312 is pushed into the first shell 310, as shown in FIG. 5, the first end 406 of the second shell 312 engages the first stop 430. As such, in the compressed state, a portion of the second shell 312 still extends outward from the second end 318 of the first shell 310. Similarly, as shown in FIGS. 4 and 5, a second stop 432 is disposed on (coupled to) an inner surface of the second shell 312. In this example, the second stop 432 is also implemented as ring. When the third shell 314 is pushed into the second shell 312, as shown in FIG. 5, the first end 410 of the third shell 314 engages the second stop 432. As such, in the compressed state, a portion of the third shell 314 still extends outward from the second end 320 of the second shell 312. Similarly, as shown in FIGS. 4 and 5, a third stop 434 is disposed on (coupled to) an inner surface of the third shell 314. In this examples, the third stop 434 is also implemented as ring. When the electrode pin 316 is pushed into the third shell 314, as shown in FIG. 5, the first end 414 of the electrode pin 316 engages the third stop 434. As such, in the compressed state, a portion of the electrode pin 316 (e.g., the electrode tip 324) extends outward from the second end 322 of the third shell 314. In an example operation, assume the electrode 302 is in the extended state. When the electrode tip 324 contacts the scalp of a subject, the electrode pin 316 is pushed upward (e.g., against the bias of the spring 416) into the third shell 314. When the first end 414 of the electrode pin 316 engages the third stop 434, the electrode pin 316 pushes the third shell 314 into the second shell 312. When the first end 410 of the third shell 314 engages the second stop 432, the third shell 314 pushes the second shell 312 into the first shell 310, until the first end 406 of the second shell 312 engages the first stop 430. While in this example, the first, second, and third stops 430, 432, 434 are implemented as rings, in other examples the first, second, and/or third stops 430, 432, 434 may be implemented as other structures, such as one or more protrusions or tabs.

In other examples, instead of using stops, the lengths of the shells 310, 312, 314 and the electrode pin 316 may be dimensioned such that when the electrode 302 is in the compressed state, a portion of one or more of the shells 310, 312, 314 and/or the electrode pin 316 extends outward. For example, a length of the second shell 312 may be greater than a length of the first shell 310, such that if the second shell 312 is fully pushed into the first shell 310 and engages the first end 402 of the first shell 310, a portion of the second shell 312 still extends outward from the second end 318 of the first shell 310. The third shell 314 may be longer than the second shell 312, and the electrode pin 316 may be longer than the third shell 314. In other examples, in the compressed state, the second end 322 of the third shell 314 may be moved flush or even with the second end 320 of the second shell 312 and/or the second end 320 of the second shell 312 may be moved flushed or even with the second end 318 of the first shell 310.

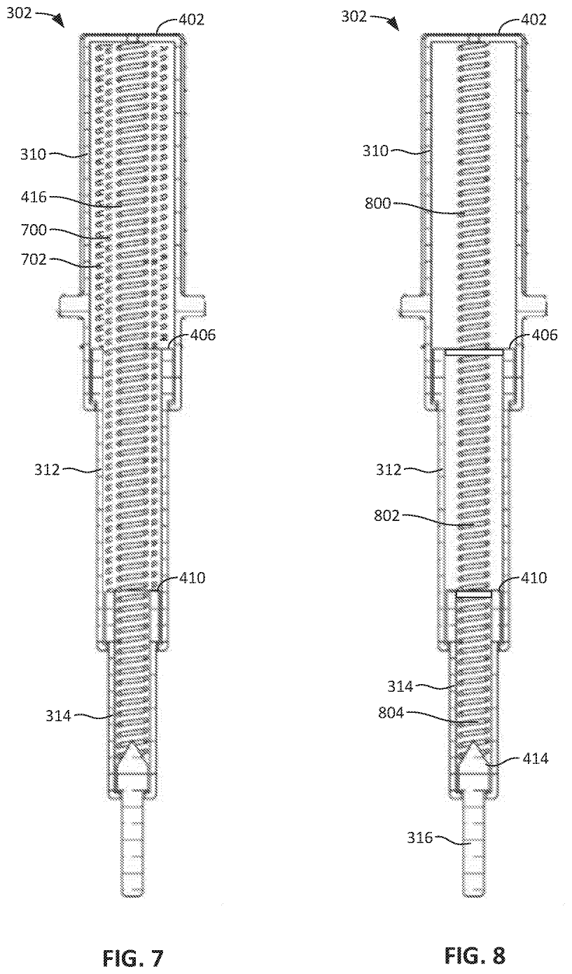

To bias the electrode 302 to the expanded position, the example electrode 302 may include one or more springs. As shown in the example of FIGS. 4 and 5, the example electrode 302 includes a spring 416. In some examples, the spring 416 provides 0.4 Newtons (N) of force, which is sufficient to gather quality signals while still providing comfort. In other examples, the spring 416 may be sized to provide more or less force. The example spring 416 is a compression coil spring. In other examples, other types of springs may be implemented. In the illustrated example, the spring 416 is disposed within and extends through the first shell 310, the second shell 312, and the third shell 314. The spring 416 extends between the first end 402 of the first shell 310 and the first end 414 of the electrode pin 316. In the illustrated example, the first end 414 of the electrode pin 316 is tapered, such that it extends into the spring 416 to help hold and position (e.g., center) the spring 416. The spring 416 biases the electrode pin 316 outward, which biases the third shell 314 outward from the second shell 312, and which biases the second shell 312 outward from the first shell 310. The spring 416 enables the electrode pin 316 to retract into the first, second, and/or third shells 310, 312, 314 when force is applied downward against the scalp. This increases the comfort and wearability of a headset (e.g., the headset 100 of FIG. 1) by reducing the pressure the electrode 302 applies to the scalp. In the illustrated example, the first shell 310, the second shell 312, the third shell 314, and the electrode pin 316 are aligned along a common axis (e.g., a central or longitudinal axis of the housing 304). In other examples, one or more additional spring(s) may be included and/or the spring(s) may be arranged in other configurations. Other example configurations of springs are disclosed in connection with FIGS. 7 and 8.

The shells 310-314 and the electrode pin 316 form a relatively long, thin electrode that is capable of protruding through longer hair, thicker hair, and/or curly hair such as afro-textured hair. Thus, the example electrode 302 is capable of obtaining better signals than other electrodes that do not effectively penetrate these hair types. While in the illustrated example of FIGS. 3-5 the electrode 302 includes three shells and one electrode pin, in other examples, the electrode 302 may include more (e.g., four, five, etc.) or fewer (e.g., one or two) shell(s).

Referring back to FIGS. 3 and 4, to couple the electrode unit 300 to one of the connectors 116a-116n of the headset 100 (FIG. 1), the example electrode unit 300 includes a connector 328. The connector 328 is disposed in an opening 418 (FIG. 4) in the second side 308 of the housing 304 that defines the cavity 400. In some examples, the connector 328 is disposed within and coupled to the electrode housing 304 via an interference fit (sometimes referred to as friction or press fit). Additionally or alternatively, in some examples a chemical fastener such as an adhesive and/or a mechanical fastener(s) may be used to couple the connector 328 to the housing 304. As shown in FIG. 4, the connector 328 includes an opening 420. The electrode 302 extends through the opening 420 of the connector 328 and outward from the housing 304.

In the illustrated example, the connector 328 is magnetic. As such, the electrode unit 300 may be magnetically coupled to one of the bands, such as the first band 106. For example, the connector 328 may be a magnetic ring and the first connector 116a of FIG. 2 may be a metal ring. Therefore, when the electrode unit 300 is placed on the first band 106 at the first aperture 114a, the first connector 116a and the connector 328 magnetically couple the electrode unit 300 to the first band 106. In other examples, the first connector 116a may be a magnetic ring, and the connector 328 of the electrode unit 300 may be metal ring. In still other examples, both the first connector 116a and the connector 328 may be magnetic rings. The magnetic attraction between the connector 328 and the first connector 116a is strong enough to hold the electrode unit 300 on the first band 106, but also enables a user to grab and pull the electrode unit 300 off of the first band 106 by overcoming the magnetic force. The magnetic coupling (or other releasable mechanical coupling) enables multiple ones of the electrode unit 300 to be independently coupled to the headset 100. As such, the multiple ones of the electrode unit 300 can be repaired or replaced individually, which extends the useful life of the headset 100 by enabling replacement of individual electrode units instead of the entire headset 100.

In the illustrated example, the connector 328 has a first side 422 (FIG. 4) (e.g., a top side) and a second side 330 (e.g., a bottom side) opposite the first side 422. As shown in FIG. 4, the first side 422 is engaged with a ledge 424 formed in the opening 418 in the housing 304. In the illustrated example, the second side 330 of the connector 328 extends beyond or is spaced apart from the second side 308 of the housing 304, which enables the connector 328 to engage the corresponding connector on the headset 100. In other examples, the second side 330 of the connector 328 may be substantially flush or even with the second side 308 of the housing 304.

In some examples, the first shell 310 (and, thus, the electrode 302) is removably disposed within the cavity 400 of the housing 304. As shown in FIG. 4, to couple the electrode 302 to the housing 304, the electrode 302 includes a flange 426 extending outward from the first shell 310. In the illustrated example, the flange 426 is clamped between (e.g., sandwiched between) the first side 422 of the connector 328 and a second ledge 428 formed in the opening 418 in the housing 404. In some examples, the electrode 300 is also disposed within and coupled to the electrode housing 304 via an interference fit (sometimes referred to as friction or press fit). Additionally or alternatively, in some examples a chemical fastener such as an adhesive and/or a mechanical fastener(s) may be used to couple the electrode 302 to the housing 304.

The electrode 302 senses EEG signals from the scalp of a subject. In particular, EEG signals are sensed at the electrode pin 316 (which may be in contact with or close to the scalp), which are transferred through the third shell 314, the second shell 312, and the first shell 310. The signals are transferred from the first shell 310 to the connector 328 (e.g., via the flange 426), and from the connector 328 to the corresponding connector on the headset, such as the first connector 116a. In some examples, as disclosed above, a PCB may be disposed within the first band 106 to electrically couple the first connector 116a to the processor 120. In some examples, the electrode pin 316, the shells 310-314, and the connector 328 are electrically conductive, whereas the housing 304 is not electrically conductive. For example, the electrode 302 may be constructed of a metallic material (e.g., steel, silver, silver-chloride, chromium, gold, etc.) and the housing 304 may be plastic or rubber and/or other suitable material(s) or combination of material(s).

FIG. 6 is an exploded view of the example electrode unit 300 showing the electrode 302, the housing 304, and the connector 328. As shown in FIG. 6, the electrode 302, the housing 304, and the connector 328 have a circular cross-section. In other examples, the electrode 302, the housing 304, and/or the connector 328 may be shaped differently. When the electrode unit 300 is assembled, the connector 328 and the electrode 302 are disposed within the housing 304, and the electrode 302 extends through the opening 420 in the connector 328.

FIG. 7 shows an alternative spring configuration that may be implemented in connection with the example electrode 302. In this example, the electrode 302 includes a second spring 700 and a third spring 702. The second spring 700 is concentric with the spring 416 (referred to as the first spring 416). The second spring 700 extends between the first end 402 of the first shell 310 and the first end 410 of the third shell 314. The second spring 700 biases the third shell 314 outward. Similarly, the third spring 702 is concentric with the first spring 416 and the second spring 700. The third spring 702 extends between the first end 402 of the first shell 310 and the first end 406 of the second shell 312. The third spring 702 biases the second shell 312 outward. In some examples, using these additional springs creates a progressively increasing force as the electrode 302 is compressed.

FIG. 8 shows another spring configuration that may be implemented in connection with the example electrode 302. In FIG. 8, the first end 406 of the second shell 312 is closed or substantially closed, and the first end 410 of the third shell 314 is closed or substantially closed. A first spring 800 is disposed within the first shell 310 and extends between the first end 402 of the first shell 310 and the first end 406 of the second shell 312. The first spring 800 biases the second shell 312 outward from the first shell 310. A second spring 802 is disposed within the second shell 312. The second spring 802 extends between the first end 406 of the second shell 312 and the first end 410 of the third shell 314. The second spring 802 biases the third shell 314 outward from the second shell 312. A third spring 804 is disposed within the third shell 314. The third spring 804 extends between the first end 410 of the third shell 314 and the first end 414 of the electrode pin 316. The third spring 804 biases the electrode pin 316 outward from the third shell 314.

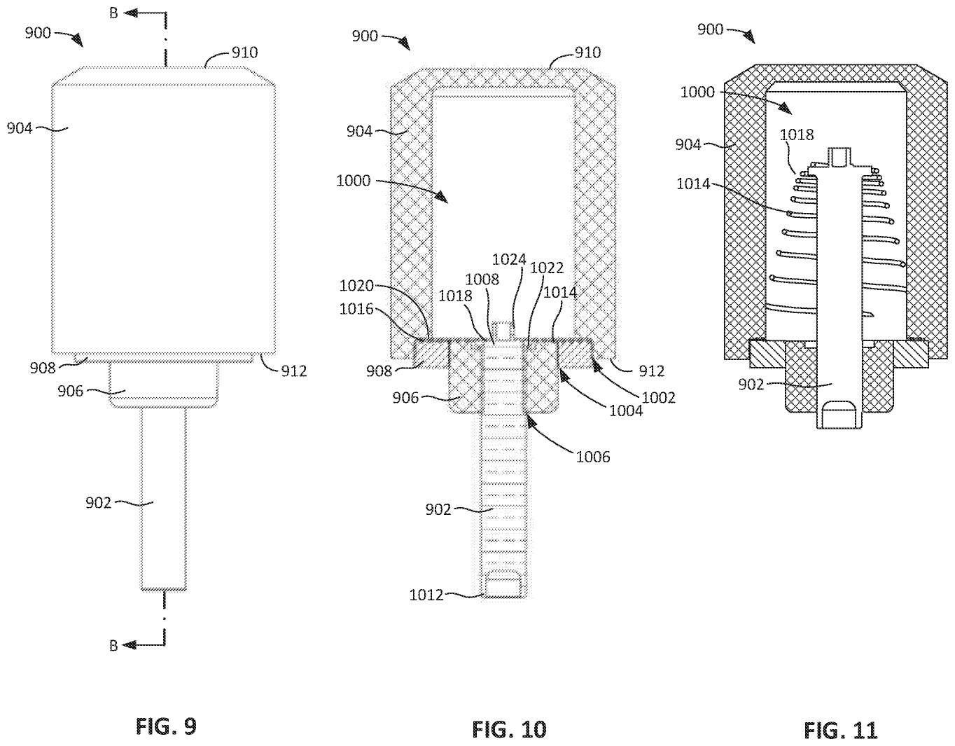

FIG. 9 illustrates another example electrode unit 900 having an example electrode 902 that may be used with the example headset 100 of FIG. 1 or another headset. FIG. 10 is a cross-sectional view of the example electrode unit 900 taken along line B-B of FIG. 9.

In the illustrated example of FIGS. 9 and 10, the electrode unit 900 includes a housing 904, the electrode 902 (which may be referred to as a plunger), a guide 906, and a connector 908. The housing 904 has a first side 910 (e.g., a top side) and a second side 912 (e.g., a bottom side) opposite the first side 910. Similar to the electrode unit 300 disclosed above, the electrode 902 of the example electrode unit 900 is coupled to the housing 904 and extends outward from the second side 912 of the housing 904. In some examples, the housing 904 is constructed of a non-conductive material, such as plastic, to help block or attenuate noise and other interference from affecting the signals gathered by the electrode 902. The electrode unit 900 may be disposed on one of the bands 104-112 of the headset 100 at one of the apertures 114a-114n, such as the first aperture 114a in the first band 106 shown in FIG. 2, for example. When the electrode unit 900 is disposed on the first band 106, for example, the electrode 902 extends through the first aperture 114a to engage the head 102 of the subject. The electrode unit 900 may be coupled to the first band 106 via the connector 908, as disclosed in further detail herein. The electrode 902 is movable into and out of the housing 904 between an extended or expanded state, as shown in FIGS. 9 and 10, and a retracted or compressed state, as shown in FIG. 11.

As shown in FIG. 10, the housing 904 has a cavity 1000 defined by an opening 1002 formed in the second side 912 of the housing 904. The connector 908 is disposed within the opening 1002. In some examples, the connector 908 is disposed within and coupled to the electrode housing 304 via an interference fit. Additionally or alternatively, in some examples a chemical fastener such as an adhesive and/or a mechanical fastener(s) may be used to couple the connector 908 to the housing 904. The connector 908 includes an opening 1004. The guide 906 is dimensioned to be disposed within the opening 1004 in the connector 908. In some examples, the guide 906 is coupled to the connector 908 via an interference fit. Additionally or alternatively, in some examples a chemical fastener such as an adhesive and/or a mechanical fastener(s) may be used to couple the guide 906 to the connector 908.

In the illustrated example, the guide 906 defines an opening 1006. The electrode 902 is slidably disposed within the opening 1006 of the guide. The electrode 902 is moveable through the guide 906 and into the cavity 1000 in the housing 904. The electrode 902 has a first end 1008 (e.g., a top end) and a second end or tip 1010 opposite the first end. In the illustrated example, the tip 1012 has an example hollow shape. An example of the hollow shape tip is disclosed in connection with FIG. 16. In other examples, the tip 1012 can be shaped differently.

To bias the electrode 902 outward from the housing 904, the example electrode unit 900 includes a spring 1014, as shown in FIG. 10. In this example, the spring 1014 is a conical compression or extension spring. The spring 1014 is disposed within the cavity 1000 of the housing 904. In the illustrated example, an outer portion 1016 of the spring is coupled to the housing 904, and an inner portion 1018 of the spring 1014 is coupled to the electrode 902. In particular, in this example, the outer portion 1016 of the spring 1014 is clamped (e.g., retained) between the connector 908 and a ledge 1020 formed in the opening 1002 in the housing 904. The inner portion 1018 of the spring 1014 is coupled to the first end 1008 of the electrode 902. In the illustrated example, the first end 1008 of the electrode 902 includes a flange 1022 and a post 1024. The flange 1022 has a larger diameter than the opening 1006 in the guide 906, thereby preventing the electrode 902 from being completely ejected from the guide 906. The post 1024 extends through the inner portion 1018 of the spring 1014 to help center and retain the spring 1014.

When the electrode 902 is in the extended position, as shown in in FIG. 10, the spring 1014 is substantially flat and lies over the guide 906 and the connector 908. The electrode 902 is retractable into the cavity 1000 of the housing 904. For example, when the electrode 902 contacts the scalp, the electrode 902 may be pushed upward into the cavity 1000 of the housing 904, as shown in FIG. 11. When the electrode 902 is moved into the cavity 1000, the inner portion 1018 of the spring 1014 is flexed upward into the cavity 1000. The spring 1014, thus, softens the force of the electrode 902 against the scalp and increases comfort to the subject wearing a headset because the spring 1014 enables the electrode 902 to retract into the housing 904.

Similar to the connector 328 disclosed above, the connector 908 may be a magnetic ring or a metallic ring to couple with a corresponding one of the connectors 116a-116n on the headset. In the illustrated example, the connector 908 extends outward slightly from the second side 912 of the housing 904. In other examples, the connector 908 may be substantially flush or even with the second side 912 of the housing 904.

Signals gathered by the electrode 902 are transmitted through the spring 1014 to the connector 908, and from the connector 908 to the corresponding connector on the headset. In some examples, the electrode 902 may be constructed of a metallic material (e.g., steel, silver, silver-chloride, chromium, gold, etc.), whereas the guide 906 and the housing 904 may be plastic or rubber and/or other suitable material(s) or combination of material(s). In other examples, the guide 906 may also be constructed of a metallic material, in which case the signals may be transmitted through the guide 906 to the connector 908.

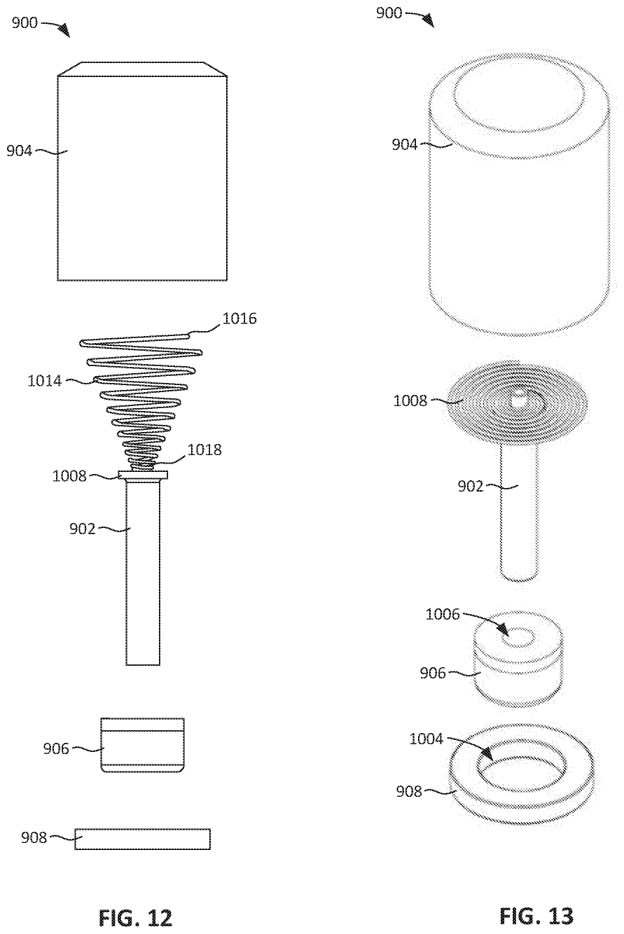

FIG. 12 is an exploded side view of the example electrode unit 900. As shown in FIG. 12, the electrode unit 900 includes the electrode 902, the housing 904, the guide 906, the connector 908, and the spring 1014. In an unbiased or relaxed position, the spring 1014 extends upward from the first end 1008 of the electrode 902. The term “upward” is used here in the manner described above. When the electrode unit 900 is assembled, as shown in FIG. 10, the inner portion 1018 of the spring 1014 is pushed upward to a position where the inner portion 1018 is substantially flush or even with the outer portion 1016 of the spring 1014. Thus, when the electrode unit 900 is assembled, the spring 1014 is partially compressed. When the electrode 902 is pushed into the housing 904, as shown in the position in FIG. 11, the inner portion 1018 of the spring 1014 is pushed through the outer portion 1016 of the spring 1014, in the opposite direction of the unbiased or relaxed position shown in FIG. 12. By using a spring with an unbiased or relaxed position as shown in FIG. 12, when the electrode unit 900 is assembled, the spring 1014 is partially compressed and, thus, provides a biasing force when the electrode 902 is in the extended position. This ensures the electrode 902 is biased in the extended position and provides sufficient force to make solid contact with the scalp.

FIG. 13 is an exploded perspective view of the example electrode unit 900 showing the electrode 902, the housing 904, the guide 906, the connector 908, and the spring 1014. In FIG. 13, the spring 1014 is shown in the partially compressed state, which is the state of the spring 1014 when the electrode unit 900 is assembled and the electrode 902 is in the extended position. However, in other examples, the unbiased or relaxed state of the spring 1014 may be substantially flat as shown in FIG. 13. When assembled, the electrode 902 is moveable through the opening 1006 in the guide 906. The guide 906 is disposed in the opening 1004 and coupled to the connector 908.

FIGS. 14-16 illustrate example electrode tips that may be implemented on the electrode pin 316 of FIG. 3, the electrode 902 of FIG. 9, and/or any other electrode. FIG. 14 is a side view of a bottom or distal portion of an electrode 1400 intended to contact the scalp of a user to obtain EEG signals. The electrode 1400 may correspond to the electrode pin 316 of the example electrode 302 of FIG. 3 or the electrode 902 of FIG. 9. The electrode 1400 has a circular cross section. In this example, an end or tip 1402 of the electrode 1400 has an end surface 1404 that is substantially flat (not convex or concave). As such, a relatively large contact area may be formed between the end surface 1404 of the electrode 1400 and the scalp or tissue on the head of the user. The end surface 1404 is perpendicular to a side surface 1406 of the electrode 1400. In this example, an edge 1408 between the end surface 1404 and the side surface 1406 is rounded or curved to reduce sharp edges or corners that could inflict pain or discomfort on the user. In other examples, the radius of the edge 1408 may be greater or less than shown in FIG. 14. In still other examples, the edge 1408 may not be curved or rounded.

FIG. 15 is a side view of a bottom or distal portion of an electrode 1500 intended to contact the scalp of a user to obtain EEG signals. The electrode 1500 may correspond to the electrode pin 316 of the example electrode 302 of FIG. 3 or the electrode 902 of FIG. 9. The electrode 1500 has a circular cross section. In this example, an end or tip 1502 of the electrode 1500 has a hump shape. In particular, the tip 1502 has a first end surface 1504 that is substantially flat or planar (not convex or concave). The first end surface 1504 is perpendicular to a side surface 1506 of the electrode 1500. The tip 1502 includes a hump 1508 extending from a center of the first end surface 1504, such that the first end surface 1504 surrounds the hump 1508. The hump 1508 is defined by a side surface 1510 and a second end surface 1512. The second end surface 1512 is substantially flat or planar (not convex or concave). The second end surface 1512 is parallel to and spaced apart from the first end surface 1504. While in the illustrated example the side surface 1510 of the hump 1508 appears curved, in some examples, at least a portion of the side surface 1510 of the hump 1508 is parallel to the side surface 1506 of the electrode 1500. In the illustrated example, the hump 1508 extends a distance D from the first end surface 1504. In other words, the second end surface 1512 is separated from the first end surface 1504 by the distance D. The hump 1508 has a width W (diameter). In some examples, the distance D is about 0.4 millimeters (mm) (e.g., ±0.02 mm) and the width W is about 0.8 mm (e.g., ±0.02 mm). In some examples, these dimensions result in strong signal quality while still providing comfort. In other examples, the distance D may be larger (e.g., 0.5 mm, 0.6 mm, etc.) or smaller (e.g., 0.3 mm, 0.2 mm, etc.) and/or the width W may be larger (e.g., 0.9 mm, 1.0 mm, etc.) or smaller (e.g., 0.7 mm, 0.6 mm, etc.).

The hump 1508 has a smaller diameter or cross-section than the rest of the electrode 1500 (at the side surface 1506), which enables the hump 1508 to extend further through the hair than the rest of the electrode 1500. Further, the smaller cross-section enables the hump 1508 to penetrate through or compress into the first layer of skin on the scalp, such as the stratum corneum. By compressing into the first layer of skin, the electrode 1500 can obtain better quality signals. However, the first end surface 1504 prevents the entire electrode 1500 from being pressed too far into the scalp. Thus, the hump shape tip enables lower impedance (and, thus, better signals) without comprising comfort.

In some examples, to increase comfort, the edges of the tip 1502 may be rounded or curved. For example, an edge 1514 between the first end surface 1504 and the side surface 1506 is rounded or curved. A corner 1516 between the first end surface 1504 and the side surface 1510 of the hump 1508 is rounded or curved. Further, an edge 1518 between the side surface 1510 of the hump 1508 and the second end surface 1512 of the hump 1508 is rounded or curved. In other examples, the radius of any of the edges 1514, 1518 and/or the corner 1516 may be greater or less than shown. In still other examples, the edges 1514, 1518 and/or the corner 1516 may not be rounded or curved.

FIG. 16 is a side cross-sectional view of a bottom or distal portion of an electrode 1600 intended to contact the scalp of a user to obtain EEG signals. The electrode 1600 may correspond to the electrode pin 316 of the example electrode 302 of FIG. 3 or the electrode 902 of FIG. 9. The electrode 1600 has a circular cross-section. In this example, an end or tip 1602 of the electrode 1600 has a hollow shape. For example, the tip 1602 has an end surface 1604 with a recess 1606 (e.g., a bore, a groove, a cavity) is formed in a center of the end surface 1604. As a result, the end surface 1604 forms a ring-shaped surface that surrounds the recess 1606. The recess 1606 is defined by a side surface 1608 and a bottom surface 1610. In the illustrated example, the end surface 1604 is substantially flat or even (not convex or concave). The end surface 1604 may be perpendicular to a side surface 1612 of the electrode 1600 and/or the side surface 1608 of the recess 1606. In some examples, a width (W) of the wall defined between the side surface 1612 of the electrode 1600 and the side surface 1608 of the recess 1606 is about 0.3 mm (e.g., ±0.02), which ensure a comfortable contact while still obtaining quality signals. In other examples, the width W may be larger (e.g., 0.4 mm, 0.5 mm, etc.) or smaller (e.g., 0.2 mm, 0.1 mm, etc.).

In the illustrated example, the recess 1606 does not extend all the way through the center of the electrode 1600. In other words, the electrode 1600 is not completely hollow or tube-shaped. In some examples, the recess 1606 extends a certain depth (D) into the end surface 1604, such as about 1 mm to about 1.5 mm (e.g., ±0.02 mm). This prevents the electrode 1600 from being stabbed into the scalp of the head. In other words, as the electrode 1600 is pressed against the scalp of the user, the end surface 1604 may penetrate or compress slightly into the first layer of skin. However, eventually the bottom surface 1610 of the recess 1606 engages the skin, which increases the contact area and prevents the electrode 1600 from being pushed further into the skin. In other examples, the depth D of the recess 1606 may be greater or smaller.

In some examples, to increase comfort, the edges of the electrode 1600 may be rounded or curved. For example, an edge 1614 (e.g., an outer edge) between the end surface 1604 and the side surface 1612 may be rounded or curved. Further, an edge 1616 (e.g., an inner edge) between the end surface 1604 and the side surface 1608 of the recess 1606 may also be rounded or curved. In some examples, a corner 1618 between the side surface 1608 and the bottom surface 1610 of the recess 1606 is rounded or curved. In other examples, the radius of any of the edges 1614, 1616 and/or the corner 1618 may be greater or less than shown. In still other examples, the edges 1614, 1616 and/or the corner 1618 may not be rounded or curved. While the edges 1614, 1616 on either side of the end surface 1604 may be curved, the end surface 1604 is still flat or even to provide a contact area with the scalp.

In some examples, the example hollow shape shown in FIG. 16 is advantageous for use with bald heads and/or along the temple area of a head where little or no hair is found. In general, dry electrodes are pressure dependent. To obtain lower impedance, it is desirable to have less contact area and/or greater pressure. From a comfort perspective, it is desirable to have a larger contact surface area. However, to obtain lower impedance from a larger contact surface area, more force has been applied. The example hollow shape of FIG. 16 enables the distribution of force on a larger area with less surface contact, thereby resulting in greater comfort without compromising signal (impedance) quality.

In some examples, a combination of the electrode tips 1400, 1500, 1600 may be used on the same headset. For example, the electrode 1500 with the tip 1502 having the hump 1508 may be used in the parietal and occipital regions of the head. In addition, the electrode 1600 with the tip 1602 have the hollow shape may be used in the frontal and temporal regions of the head. In some examples, a system can alert a user (e.g., the subject) to switch one type of electrode tip for another type of electrode tip based on impedance. For example, the processor 120 (FIG. 1) and/or another electronic device can measure the impedance of one of the electrodes. Based on the impedance, the processor 120 (and/or another electronic device) generates a message or alert (e.g., via a message on a screen of a computer) to switch the current electrode and/or electrode unit to a different electrode and/or electrode unit having a different tip. For example, if a flat electrode tip is being used and impedance is relatively high (e.g., based on a comparison to an impedance threshold), the processor 120 may signal to the user to switch to another type of electrode tip, such as the hump shape electrode tip or a hollow shape electrode tip.

From the foregoing, it will be appreciated that example electrodes and example electrode units having electrodes have been disclosed that are capable of obtaining between quality signals. Example telescoping electrodes disclosed herein can penetrate longer, thicker, and/or curly hair to make solid contact with the scalp. Example electrode tip shapes have also been disclosed that provide better contact with the scalp to obtain stronger signals without reducing comfort.

Example electrodes and electrode units for gathering EEG data are disclosed herein. Further examples and combinations thereof include the following:

An example electrode unit disclosed herein includes a housing and an electrode coupled to and extending from the housing. The electrode includes a first shell, a second shell retractable relative to the first shell, a third shell retractable relative to the second shell, and an electrode pin retractable relative to the third shell. The first shell, the second shell, the third shell, and the electrode pin are configured in a telescoping arrangement and moveable between an expanded state and a compressed state. The electrode includes a spring to bias the second shell outward from the first shell, the third shell outward from the second shell, and the electrode pin outward from the third shell to contact skin of a subject.

In some examples, the spring extends through the first shell, the second shell, and the third shell. In some such examples, the spring extends between a top end of the first shell and a top end of the electrode pin. In some examples, the electrode pin has a tip, opposite the top end of the electrode pin, that is to contact the skin of the subject. In some examples, the top end of the electrode pin is tapered. In some examples, the spring is a first spring, the electrode further includes a second spring. The second spring extends between the top end of the first shell and a top end of the third shell. The second spring is to bias the third shell outward. In some such examples, the electrode further includes a third spring. The third spring extends between the top end of the first shell and a top end of the second shell. The third spring is to bias the second shell outward. In some examples, the first, second, and third springs are concentric.

In some examples, the third shell includes a stop that is engaged by the electrode pin in the compressed state, such that a portion of the electrode pin extends outward from an end of the third shell. In some examples, the housing includes an opening defining a cavity. The first shell is removably disposed in the cavity. The first shell has a flange that is clamped between a connector and a ledge formed in the opening in the housing. In some examples, a tip of the electrode pin has an end surface with a recess extending into the end surface such that the end surface forms a ring around the recess. The end surface is substantially flat. In some examples, a tip of the electrode pin has a first end surface being perpendicular to a side surface of the electrode pin. The tip has a hump extending from the first end surface such that the first end surface surrounds the hump. The hump having a second end surface spaced apart from the first end surface.

An example electrode unit disclosed herein includes a housing and an electrode extending from the housing. The electrode has a first end surface, a side surface perpendicular to the end surface, and a hump extending from the first end surface such that the first end surface surrounds the hump. The hump has a second end surface spaced apart from the first end surface.

In some examples, the hump extends from the first end surface about 0.4 millimeters. In some examples, the second end surface is parallel to the first end surface. In some examples, an edge between the second end surface and a side surface of the hump is rounded.

An example electrode unit disclosed herein includes a housing and an electrode extending from the housing. The electrode has a tip with an end surface and a recess extending into the end surface such that the end surface forms a ring around the recess. The end surface is substantially flat.

In some examples, an outer edge between the end surface and a side surface of the electrode is rounded. In some such examples, an inner edge between the end surface and a side surface that defines the recess is rounded.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An electrode unit comprising:
a housing; and
an electrode coupled to and extending from the housing, the electrode including a first shell, a second shell retractable relative to the first shell, a third shell retractable relative to the second shell, and an electrode pin retractable relative to the third shell, the first shell, the second shell, the third shell, and the electrode pin configured in a telescoping arrangement and moveable between an expanded state and a compressed state, the electrode including a spring to bias the second shell outward from the first shell, the third shell outward from the second shell, and the electrode pin outward from the third shell to contact skin of a subject.

2. The electrode unit of claim 1, wherein the spring extends through the first shell, the second shell, and the third shell.

3. The electrode unit of claim 2, wherein the spring extends between a top end of the first shell and a top end of the electrode pin.

4. The electrode unit of claim 3, wherein the electrode pin has a tip, opposite the top end of the electrode pin, that is to contact the skin of the subject.

5. The electrode unit of claim 3, wherein the top end of the electrode pin is tapered.

6. The electrode unit of claim 3, wherein the spring is a first spring, the electrode further including a second spring, the second spring extending between the top end of the first shell and a top end of the third shell, the second spring to bias the third shell outward.

7. The electrode unit of claim 6, wherein the electrode further includes a third spring, the third spring extending between the top end of the first shell and a top end of the second shell, the third spring to bias the second shell outward.

8. The electrode unit of claim 7, wherein the first, second, and third springs are concentric.

9. The electrode unit of claim 1, wherein the third shell includes a stop that is engaged by the electrode pin in the compressed state, such that a portion of the electrode pin extends outward from an end of the third shell.

10. The electrode unit of claim 1, wherein the housing includes an opening defining a cavity, the first shell removably disposed in the cavity, the first shell having a flange, the flange being clamped between a connector and a ledge formed in the opening in the housing.

11. The electrode unit of claim 1, wherein a tip of the electrode pin has an end surface with a recess extending into the end surface such that the end surface forms a ring around the recess, the end surface being substantially flat.

12. The electrode unit of claim 1, wherein a tip of the electrode pin has a first end surface being perpendicular to a side surface of the electrode pin, the tip having a hump extending from the first end surface such that the first end surface surrounds the hump, the hump having a second end surface spaced apart from the first end surface.

13. An electrode unit comprising:
a housing; and
an electrode extending from the housing, the electrode having:
a first end surface;
a side surface perpendicular to the end surface; and
a hump extending from the first end surface such that the first end surface surrounds the hump, the hump having a second end surface spaced apart from the first end surface.

14. The electrode unit of claim 13, wherein the hump extends from the first end surface about 0.4 millimeters.

15. The electrode unit of claim 13, wherein the second end surface is parallel to the first end surface.

16. The electrode unit of claim 13, wherein an edge between the second end surface and a side surface of the hump is rounded.

17. An electrode unit comprising:
a housing; and
an electrode extending from the housing, the electrode having a tip with an end surface and a recess extending into the end surface such that the end surface forms a ring around the recess, the end surface being substantially flat.

18. The electrode unit of claim 17, wherein an outer edge between the end surface and a side surface of the electrode is rounded.

19. The electrode unit of claim 18, wherein an inner edge between the end surface and a side surface that defines the recess is rounded.
